(19) United States
(12) Patent Application Publication
(10) Pub. No.: US 2011/0092579 A1
(43) Pub. Date: Apr. 21, 2011

(54) Solubilized formulation of docetaxel

(75) Inventor: Nageswara R. Palepu, Southampton, PA (US)

(73) Assignee: Scidose LLC, Amherst, MA (US)

(21) Appl. No.: 12/589,144

(22) Filed: Oct. 19, 2009

(51) Int. Cl. A61K 31/337; A61P 1/00

(52) U.S. Cl. 514/449

(57) ABSTRACT

Docetaxel containing formulations having TPGS and being substantially free or totally free of polysorbate surfactants are disclosed wherein stability is enhanced and hypersensitivity reactions are reduced.

### CROSS-REFERENCE TO RELATED APPLICATIONS

Not applicable.

### STATEMENT REGARDING FEDERALLY SPONSORED RESEARCH OR DEVELOPMENT

Not Applicable

### FIELD OF THE INVENTION

The present invention relates liquid formulations of docetaxel, more specifically to high level concentrates of docetaxel in non-aqueous solvents, to diluents therefor (hereinafter “primary diluent”) in the preparation of intermediate concentrates, to the target concentration range for infusions for animals (inclusive of without limitation, farm animals, zoo animals, pet animals, and humans, particularly mammals and most particularly humans) made therefrom. The invention further relates to the use of tocopherol polyethylene glycol (1000) succinate (TPGS) in appropriate concentrations to minimize infusion related reactions along with ethanol and PEG 400 in the preparation of such products, so that disadvantageous materials such as polysorbates and or cremophor type materials (polyethoxylated oils, whether unhydrogenated, hydrogenated, or partially hydrogenated) can be avoided in the preparations.

### BACKGROUND OF THE INVENTION

Docetaxel is an antineoplastic agent belonging to the taxoid family being marketed by Sanofi-Aventis under trade name Taxotere®. It is prepared by semisynthesis beginning with a precursor extracted from the renewable needle biomass of yew plants. The chemical name for docetaxel is (2R,3S)-N-carboxy-3-phenylisoserine, N-tert-butyl ester, 13-ester with 5beta-20-epoxy-1,2α,4,7β,10β,13α-hexahydroxytax-11-en-9 -one 4-acetate 2-benzoate, trihydrate. Docetaxel has the following structural formula:

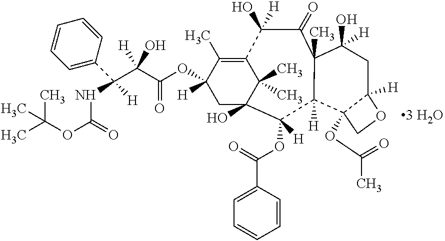

Docetaxel, as currently marketed by Sanofi-Aventis, is a white to almost-white powder with an empirical formula of C₄₃H₅₃NO₁₄.3H₂O, and a molecular weight of 861.9. It is highly lipophilic and practically insoluble in water. Taxotere® (docetaxel) Injection Concentrate is a clear yellow to brownish-yellow viscous solution. Taxotere® is sterile, non-pyrogenic, and is available in single-dose vials containing 20 mg (0.5 ml) or 80 mg (2 ml) docetaxel (on an anhydrous basis). Each ml contains 40 mg docetaxel (on an anhydrous basis) and 1040 mg polysorbate 80. For purposes of this specification, reference to an amount of “docetaxel” without reference to the specific form (i.e., hydrate, salt, etc.) will mean the stated amount of the free, anhydrous, non-solvated moiety of the drug in question unless the context clearly requires otherwise, notwithstanding the actual form of the compound then under discussion. Thus, for example, reference to 80.7 mg of docetaxel without reference to the form of the drug, means that amount of the actual drug form used which corresponds to the same number of moles of the docetaxel moiety as 80.7 mg of free, unsolvated, anhydrous docetaxel. If free docetaxel trihydrate were to be used, this would mean 86.1 mg of free docetaxel trihydrate. Similar calculations for salts and solvates will be apparent to those of ordinary skill in the art.

Taxotere® Injection Concentrate requires dilution prior to use. A sterile, non-pyrogenic, single-dose diluent is supplied for that purpose. The diluent for Taxotere® contains 13% ethanol in water for injection, and is supplied in vials. The preparation of the dilution is in two phases. The concentrate (which is stored between 2-25° C. (36 and 77° F.)) is allowed to come to room temperature, if not already, along with any necessary diluent (13% ethanol in water for injection for the commercially available material) by letting them stand under room temperature conditions for about 5 minutes. Diluent is aseptically withdrawn from its vial (approximately 1.8 ml for Taxotere®20 mg and approximately 7.1 ml for Taxotere® 80 mg) into a syringe by partially inverting the vial, and transferring it to the appropriate vial of Taxotere® Injection Concentrate. If the procedure is followed as described, an initial diluted solution of 10 mg docetaxel/ml will result. This initial dilution is mixed by repeated inversions for at least 45 seconds to assure full mixture of the concentrate and diluent. The vial should not be shaken. The resulting solution (10 mg docetaxel/ml) should be clear; however, there may be some foam on top of the solution due to the polysorbate 80. The initial diluted solution may be used immediately or stored either in the refrigerator or at room temperature for a maximum of 8 hours.

The current Taxotere label indicates that the required amount of docetaxel is then aseptically withdrawn from the initial 10 mg docetaxel/ml solution with a calibrated syringe and injected into a 250 ml infusion bag or bottle of either 0.9% Sodium Chloride solution or 5% Dextrose solution to produce a final concentration of 0.3 to 0.74 mg/ml. If a dose greater than 200 mg of Taxotere® is required, a larger volume of the infusion vehicle is used so that a concentration of 0.74 mg/ml docetaxel is not exceeded. (It has been found that if this maximum is exceeded in the final infusion concentration (using the Taxotere® formulation), the Taxotere® precipitates out of the formulation having the polysorbate as the solubilizer.) The infusion is then thoroughly mixed by manual rotation. The final Taxotere® dilution for infusion should be administered intravenously as a 1-hour infusion under ambient room temperature and lighting conditions.

Taxotere® infusion solution, if stored between 2 and 25° C. (36 and 77° F.) is stable for 4 hours. Fully prepared Taxotere® infusion solution (in either 0.9% Sodium Chloride solution or 5% Dextrose solution) should be used within 4 hours (including the 1 hour intravenous administration).

The present marketed docetaxel (in Taxotere®) is dissolved in 100% (w/v) polysorbate 80 (Tween-80) which results in severe side effects. Severe hypersensitivity reactions characterized by generalized rash/erythema, hypotension and/or bronchospasm, or very rarely fatal anaphylaxis, have been reported in patients in spite of receiving the recommended 3-day dexamethasone premedication. Hypersensitivity reactions require immediate discontinuation of the Taxotere® infusion and administration of appropriate therapy. All the hypersensitive reactions mentioned above are primarily caused by and due to the presence of polysorbate 80 in the formulation. In order to reduce the side effects induced by polysorbate 80, all patients are treated with dexamethasone for three days prior to therapy. Dexamethasone is a steroid which suppresses the immune-response in patients. Cancer patients under chemotherapy generally have a low level of immunity due to the destruction of healthy cells by the chemotherapeutic agents. Treatment with steroids will further compromise the patient's immunity and patients will be susceptible to bacterial and fungal attacks. Due to these side effects, most of the patients drop out of docetaxel therapy by the end of 2^{nd} or 3^{rd} cycle or skip a dose or continue further therapy at reduced dose. The recommended therapy is 6 cycles of docetaxel given once every three weeks. Thus, therapeutic activity and the maximum tolerated dose (MTD) of docetaxel are compromised due to the presence of polysorbate 80 in the formulation. Other solubilizing agents such as Cremophor EL (used in connection with the marketed paclitaxel product Taxol®) having similar allergic reactions (requiring pre-medication with steroids and antihistamines) should be avoided.

### OBJECTS OF THE INVENTION

It is therefore an object of the invention to provide a docetaxel formulation suitable for injection with little or no polysorbate 80 surfactant.

Yet another object of the invention is to provide a docetaxel liquid concentrate formulation that has little or no polysorbate 80 surfactant and further has little or no Cremophor surfactant.

Still another object of the invention is to provide a docetaxel liquid concentrate that has little or no polysorbate.

Another object of the invention is to provide a docetaxel liquid concentrate that has both little or no polysorbate and little or no Cremophor component.

Still another object of the invention is to provide a docetaxel liquid concentrate that is completely free of polysorbate components.

An even further embodiment of the invention is to provide a docetaxel liquid concentrate that is completely free of both polysorbate and Cremophor components.

It is a further object of the invention to provide a docetaxel (or pharmaceutically acceptable salt thereof) formulation containing TPGS at a substantially reduced level as compared to formulations specifically disclosed in US 2008/0319048.

It is yet another object of the invention to provide a docetaxel formulation that has fewer hypersensitivity reactions than the currently commercially available formulations, which currently available formulations have a polysorbate 80 surfactant component.

It is yet another object of the invention to provide a docetaxel formulation that has fewer hypersensitivity reactions than the currently commercially available formulations, which currently available formulations have a polysorbate surfactant component.

Still another object of the invention is to provide a substantially polysorbate-free docetaxel liquid concentrate formulation that is also substantially free of hydroxyalkyl-substituted cellulosic polymers.

An even further object of the invention is to provide a substantially polysorbate-free and substantially Cremophor-free docetaxel liquid concentrate formulation that is free of hydroxyalkyl-substituted cellulosic polymers.

Still another object of the invention is to provide a substantially polysorbate-free docetaxel liquid concentrate formulation that is also substantially free of substituted cellulosic polymers.

An even further object of the invention is to provide a substantially polysorbate-free and substantially Cremophor-free docetaxel liquid concentrate formulation that is free of substituted cellulosic polymers.

Still another object of the invention is to provide a substantially polysorbate-free docetaxel liquid concentrate formulation that is also substantially free of cellulosic polymers.

An even further object of the invention is to provide a substantially polysorbate-free and substantially Cremophor-free docetaxel liquid concentrate formulation that is free of cellulosic polymers.

Still another object of the invention is to provide a suitable primary dilution formulation for use in preparing the aforementioned docetaxel liquid concentrates.

An even further object of the invention is to provide a final dilution for injection of a docetaxel containing product in the substantial absence or in the total absence of polysorbate 80 surfactant.

An even further object of the invention is to provide a final dilution for injection of a docetaxel containing product in the substantial absence or in the total absence of polysorbate 80 and in the substantial absence of Cremophor.

An even further object of the invention is to provide a final dilution for injection of a docetaxel containing product in the substantial absence or in the total absence of polysorbate 80 surfactant, in the substantial or total absence of Cremophor, and in the substantial or total absence of a hydroxyalkyl-substituted cellulosic polymer.

An even further object of the invention is to provide a final dilution for injection of a docetaxel containing product in the substantial absence or in the total absence of polysorbate surfactant.

An even further object of the invention is to provide a final dilution for injection of a docetaxel containing product in the substantial absence or in the total absence of polysorbate and in the substantial absence of Cremophor.

An even further object of the invention is to provide a final dilution for injection of a docetaxel containing product in the substantial absence or in the total absence of polysorbate surfactant, in the substantial or total absence of Cremophor, and in the substantial or total absence of a hydroxyalkyl-substituted cellulosic polymer.

It is a further object of the invention to provide a a final dilution for injection docetaxel (or pharmaceutically acceptable salt thereof) formulation containing TPGS at a substantially reduced level as compared to formulations specifically disclosed in US 2008/0319048.

Still another object of the invention is to provide a suitable primary dilution for use in preparing the aforementioned final dilution for injection formulations of docetaxel.

Yet another object of the invention is to provide formulations, liquid concentrates, etc. containing docetaxel that are substantially free or totally free of any cellulosic polymer and can be reconstituted or diluted without the use of a substantial amount of or without the use of any amount of a cellulosic polymer.

Another object of the invention is to provide a means to administer docetaxel to patients without the need for administering dexamethasone or any other steroid and/or without the need to administer an antihistamine prior to the initiation of the docetaxel administration.

Yet another object of the invention is the avoidance of diarrheal side effect accompanying docetaxel administration primarily, if not totally, due to the polysorbate present in currently marketed docetaxel injection products.

An even further object of the invention is to provide a means to administer docetaxel to patients without the need for administering dexamethasone or any other steroid and/or without the need to administer an antihistamine prior to the initiation of the docetaxel administration and without the need for administering dexamethasone or any other steroid or antihistamine during or after the docetaxel administration.

Yet another object of the invention is to provide docetaxel formulation containing tocopherol polyethylene glycol (1000) succinate (TPGS) with reduced hypersensitivity/infusion reactions relative to other formulations with TPGS therein.

Still another object of the invention is to provide docetaxel formulations that are sufficiently stable, physically and chemically, that they can be used over a longer time period beginning at dilution from a high concentrate to an intermediate concentrate through further dilution to an infusion concentration and further through the infusion administration time relative to the currently marketed Taxotere product.

Still further objects of the invention will be appreciated by those of ordinary skill in the art.

### BRIEF SUMMARY OF THE INVENTION

These and other objects of the invention can be achieved by a composition comprising docetaxel and (a) at least one pharmaceutically acceptable solubilizer excipient that can dissolve docetaxel in amounts of at least 55 mg/ml or (b) a mixture of pharmaceutically acceptable hydrotropes that in concert (although not individually) are capable of dissolving docetaxel in amounts of at least 55 mg/ml or (c) mixtures thereof or (d) at least one pharmaceutically acceptable solubilization excipient that can dissolve docetaxel in amounts of at least 55 mg/ml in combination with at least one pharmaceutically acceptable solubilization aid where the solubilization aid does not alone or in combination with other solubilization aids dissolve docetaxel in amounts of at least 55 mg/ml. These docetaxel solutions are either in the pharmaceutically acceptable solubilizer, hydrotropes, or mixtures thereof directly or in water solutions thereof, generally without further solubilization aids, but further such solubilization aids may be included if desired. Two especially useful solvents for docetaxel include, without limitation, glycofurol and ethanol, as well as mixtures thereof Each of the solutions of the invention is in the substantial absence of (a) polysorbate 80, if not the total absence of polysorbate 80, (b) optionally in the substantial absence of or total absence of one or more of a polyethoxylated vegetable oil, a polyethoxylated castor oil, a polyethoxylated partially hydrogenated vegetable oil, a polyethoxylated partially hydrogenated castor oil, a polyethoxylated hydrogenated vegetable oil, a polyethoxylated hydrogenated castor oil, and (c) optionally in the substantial absence of or in the total absence of hydroxypropylmethylcellulose (preferably hydroxyalkyl alkylcellulose, more preferably substituted cellulosic polymers). The avoidance of the polysorbate 80 and Cremophor type solubilizers avoids the hypersensitivity or infusion reactions that plague existing formulations of taxanes and allows for the optional reduction or elimination of steroid and/or antihistamine pre- and/or post treatment. Avoidance of the polysorbate 80 further avoids the diarrheal side effect caused thereby. Each of these allows for better, more effective dosing regimens and better patient compliance with recommended dosings than with the currently marketed taxane injectables. The use of TPGS in order to avoid the polysorbates of the Taxotere® formulation and the avoidance of the Cremophor type materials has been disclosed in US 200/0319048, and while those formulations have been a significant improvement over the marketed Taxotere® formulation, further improvements are still required. Reducing the TPGS concentrations and glycofurol concentrations from those specifically set forth in US 2008/0319048 further lessens possible hypersensitivity reactions but results in precipitation of the docetaxel when the product is diluted with the infusion fluids to infusion administration concentrations. Surprisingly the inclusion of ethanol avoids this precipitation problem, allowing for both the reduction of these concentrations while still achieving a product that is of suitable stability, both chemically and physically, even beyond that of the Taxotere® formulation and still avoiding many of the side effects of the Taxotere® formulation. These results are especially surprising in that these reductions are reductions in solubilizing materials for the docetaxel.

### BRIEF DESCRIPTION OF THE SEVERAL VIEWS OF THE DRAWING

Not Applicable

### DETAILED DESCRIPTION OF THE INVENTION

The present invention is directed to (a) formulations of docetaxel, (b) concentrates for preparing injectable formulations of docetaxel, and further to (c) methods of manufacture of each. Methods of treatment of docetaxel treatable conditions with the docetaxel formulations, especially for treatment without the need for steroid pre-treatment or at least a reduction in the amount of steroid pre-treatment as compared to the present methods of administering docetaxel are also part of the invention as is the treatment optionally without the need for antihistamine pre/post-treatment. The formulations, concentrates, intermediate dilutions, and final administration injectable presentations are substantially free, preferably totally free of polysorbate 80, more preferably substantially free, still more preferably totally free of any polysorbate surfactant, optionally substantially free, preferably totally free, of polyethoxylated castor oil, polyethoxylated vegetable oil, their partial as well as their fully hydrogenated counterparts.

If docetaxel is formulated with non-toxic pharmaceutically acceptable excipients, it can be administered to cancer patients at much higher doses (greater than the current dosing range of 75 to 100 mg/m²), or higher infusion rates (up to at least 1 mg/ml in 10 to 15 minutes infusion time), for longer exposure to the drug (more than 6 cycles), and/or less than 3 weeks between cycles; and without missing dosing cycles or dose reduction due to side effects. In other words, if docetaxel is formulated with pharmaceutically acceptable innocuous excipients, it will be better tolerated in cancer patients and would be highly beneficial to them as they can take the medication for a longer period of time without dose interruption and reduction (and therefore potentially higher total and cumulative dose) compared to the current TAXOTERE formulation. Longer exposure to the docetaxel maintains the dose density over a longer period in the tumor and thereby helps to better eradicate the cancer cells and minimizes the relapse of the disease. Furthermore, the reduction or elimination of the steroid pre-treatment phase (in common use with the existing marketed docetaxel product) means fewer concerns with immune system depression, drug-drug interactions with other drugs which the patient may be taking, and the avoidance of side effects of steroid administration. Still further, avoidance of the Tween component (polysorbate component) means removal of a substantial cause of the diarrheal and erythema side effects seen with current docetaxel infusions. Finally, with the removal of the polysorbate component and enablement of administration at higher dosages than currently suitable, docetaxel may now be used to treat conditions which it could not previously be used to treat because of the dose limitations imposed by the polysorbate component of the current TAXOTERE formulation.

The present inventors have made efforts to develop formulations of docetaxel without the use of polysorbates and such efforts have been embodied within the disclosure of U.S. Ser. No. 12/214,506, filed Jun. 19, 2008 and published as US 2008/0319048 on Dec. 25, 2008 (the '048 Publication), the entirety of which is incorporated herein by reference. The present invention is directed to improvements over the specific disclosures therein, which included the substantial absence of ethanol, and the presence of TPGS in combination with glycofurol and docetaxel where the TPGS was present in amounts of 1500 mg TPGS/80 mg docetaxel, where the docetaxel was provided from an initial concentrate having the 80 mg of the docetaxel dissolved in glycofurol at a concentration of 40 mg docetaxel/ml. In addition, the diluents used to dilute this concentrate to 10 mg docetaxel/ml contained additional glycofurol (diluents formulations typically containing 25% glycofurol), thereby resulting in a considerable amount of glycofurol/80 mg dose. The present invention reduces both the glycofurol content of the various compositions and reduces the amount of TPGS each relative to the amount of docetaxel or pharmaceutically acceptable salt thereof as compared to the ratios presented in the '048 Publication. Surprisingly, a suitable formulation is achieved despite the substantial reduction in the primary solvent for the docetaxel (glycofurol) and the significant reduction in the primary hydrotrope in the diluent for the docetaxel concentrate. Such reductions in the solubilizing principles would have been expected to result in an unstable product, either due to lack of solubilization or the inability to maintain solubilization on dilution to infusion strength.

Glycofurol is also known as tetrahydrofurfuryl alcohol polyethylene glycol ether and has the following structure:

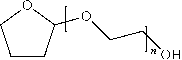

where n is on average 2 for glycofurol 75, but may be other integers for other glycofurols. Glycofurol, especially glycofurol 75, is one of the most preferred solubilizers as docetaxel is highly soluble therein (200 mg/ml in glycofurol 75). While glycofurol 75 is the most preferred of the glycofurols, those having an average n in the above formula of about 2 to about 8, preferably 2 to about 6, more preferably 2 to about 4, more preferably about 2 or about 3 or about 4 are also suitable. Larger values of n can be used, but the appropriateness of the larger glycofurols (average n in excess of about 8) falls off quickly.

Hydrotropes for the present invention are generally selected (without limitation) from the group consisting of polyethylene glycol, especially PEG 400, polyethylene glycol 600; tocopherol compounds, particularly tocopherol-polyethylene glycols, more particularly tocopherol polyethylene glycol diacid (such as succinates, maleates, etc.) esters, especially tocopherol polyethyleneglycol succinates, most preferably tocopherol polyethylene glycol 1000 succinate (TPGS 1000).

Docetaxel active agent can be dissolved in the glycofurol alone or in a mixture of the glycofurol and ethanol or in a mixture of either with the polyethylene glycol and/or the TPGS, although for stability purposes for long term storage, it is preferable to only add the TPGS and polyethylene glycol close to the time when the final dilution to infusion strength is desired, for example within about 24 hours, preferably within about 18 hours, more preferably within about 12 hours, still more preferably within about 6 hours, even more preferably within about 4 hours of when the product is to be utilized as an infusion (including the infusion administration time). The initial concentrate can be prepared in the presence or absence of water and preferably is in the absence of water. A preferred initial concentrate formulation is merely docetaxel or a pharmaceutically acceptable salt thereof in glycofurol at a concentration of 80 mg of active agent (docetaxel) based on the docetaxel moiety/ml of solution without other components. However, if desired, ethanol can be present in this concentrate up to the maximum amount of ethanol set forth below for the diluted concentrate (such as when the initial concentrate above is diluted to about 10 mg docetaxel/ml, shortly before addition to infusion fluids). In addition, if desired, the initial concentrate can further have none of, or a portion of, or all of the TPGS, polyethylene glycol, and optional tonicity agent, but preferably these latter three materials are maintained separately until dilution to the intermediate concentration amount (such as 10 mg docetaxel/ml). When some amount of the components other than the glycolfurol and docetaxel are included in the initial concentrate (concentrations of docetaxel in excess of 10 mg/ml, especially up to 40 g/ml or up to 80 mg/ml or even higher), the diluents used to dilute the initial concentrate to the intermediate concentrate (i.e., less than about 15 mg docetaxel/ ml) compensates for that amount by having a lesser amount of that same material in the diluents solution, so that upon dilution of the initial concentrate to the intermediate concentrate, and further to the infusion concentration, the ratios set forth herein between the TPGS and docetaxel and between the ethanol and the docetaxel are maintained within the stated ranges. In addition, the initial concentrate or the diluents solution further may contain an antioxidant, material, and in light of the possibility that the polyethylene glycol and the TPGS may have a slight peroxide presence therewith, the antioxidant is preferably present and preferably added to the docetaxel concurrently with or before the addition of the polyethylene glycol and the TPGS, although addition of the antioxidant slightly after these two materials is not precluded. In a very highly preferred embodiment the antioxidant material may be added to the glycofurol, most preferably at or before the addition of the docetaxel to the glycofurol. The initial high concentrate solution can be stored at room temperature or under refrigeration conditions (i.e., greater than 0° C. to about 10° C., preferably about 2-8° C. for refrigeration conditions). The initial concentrate solution is then diluted with a diluent that contains TPGS, polyethylene glycol, ethanol, water, optional antioxidant, and optional tonicity adjuster, although one or more of these may be absent or reduced in amount if the initial concentrate already has them present so that upon dilution the required levels are present in the diluted concentrate before further dilution to infusion strength. This intermediate diluted concentrate concentrate is further diluted with an injectable diluent solution or IV infusion fluids (generally water for injection, normal saline solution, or dextrose 5% for injection, although other fluids suitable for injection or infusion that are compatible with the other components of the formulation are acceptable as well) to concentrations of 0.3 to 0.74 mg/ml, for administration designed to be in the same concentration range for infusion administration as that recommended in the currently marketed Taxotere® product; however, as discussed earlier, higher infusion concentrations (at least up to 1 mg docetaxel/ml or higher) as well as faster infusion rates are also suitable for the present invention since there is no polysorbate component present. In a highly preferred embodiment, the docetaxel is dissolved in glycofurol to a concentration of about 80 mg/ml or higher and optionally (but preferably present), α-lipoic acid (or dihydrolipoic acid or the alkaline pharmaceutically acceptable salts of either or mixtures thereof, but preferably α-lipoic acid or its sodium salt or potassium salt) is also included in this initial formulation at an amount of from more than 2 mg to less than 200 mg, preferably not more than 175 mg, more preferably not more than 150 mg, still more preferably not more than 125 mg, yet more preferably not more than 100 mg, even more preferably not more than 75 mg, still more preferably not more than 50 mg, even yet more preferably not more than 25 mg, and yet more preferably not more than 20 mg, preferably from about 2.75 mg to about 15 mg, more preferably from about 3 mg to about 10 mg, still more preferably from about 4 mg to about 7.5 mg, even more preferably from about 4.5 mg to about 6 mg, and most preferably about 5mg to form a first or initial concentrate solution. In a particularly preferred embodiment of the present invention, the foregoing amounts of α-lipoic acid (or dihydrolipoic acid or the alkaline pharmaceutically acceptable salts of either or mixtures thereof) are present per ml of the initial concentrate. In another particularly preferred embodiment of the present invention, the foregoing mg amounts of α-lipoic acid (or dihydrolipoic acid or the alkaline pharmaceutically acceptable salts of either or mixtures thereof) are present per every 80 mg of docetaxel (or pharmaceutically acceptable salt thereof based on free docetaxel) that is in the initial concentrate. Separately, TPGS 1000, polyethylene glycol, ethanol, optionally (but preferably) tonicity adjuster (preferably sodium chloride) and optionally an antioxidant, (including, but not limited to, α-lipoic acid, dihydrolipoic acid, and their alkaline salts (preferably alkali metal salts, more preferably sodium or potassium salts, of either, and mixtures thereof) wherein the α-lipoic acid, dihydrolipoic acid, and their alkaline salts are included in this portion only to the extent that they are not included in the initial concentrates to the maximum amounts permitted in the initial concentrates as set forth in the preceding sentence) are dissolved in water to arrive at a diluent formulation for the initial concentrate. This liquid concentrate and the diluent solution are then packaged and stored for commercial distribution. The diluent solution is then used to dilute the docetaxel initial concentrate to intermediate diluted concentration of about 5 to about 20 mg docetaxel/ml, preferably about 8 to about 15 mg docetaxel/ml, more preferably about 10 mg docetaxel/ml. The diluted concentrate solution is then further diluted to administration concentrations (0.3 mg docetaxel/ml infusion to 0.74 mg docetaxel/ml infusion as currently recommended in the Taxotere® product label, or higher, such as up to about 1 mg docetaxel/ml infusion) with normal saline, 5% dextrose, or other suitable injection diluents for administration to the patient (i.e., the diluted concentrate is added in an appropriate amount to an infusion bag for infusion administration to a patient). In all cases, polysorbate 80 is limited to very minor amounts (substantially free of polysorbate 80), or is completely absent, preferably completely absent; more preferably any polysorbate is substantially absent and most preferably completely absent from the foregoing. In some embodiments, the liquid concentrates, the diluted concentrates, and the diluted for administration formulations are substantially free of, more preferably totally free of Cremophor, and preferably substantially free of, still more preferably totally free of all polyethoxylated vegetable oils (whether totally hydrogenated, partially hydrogenated, or not hydrogenated). In yet further embodiments, the liquid concentrates, the diluted concentrates, and the diluted for administration formulations are substantially free of, preferably totally free of hydroxyalkyl substituted cellulosic polymers (preferably substituted cellulosic polymers, more preferably cellulosic polymers). Still other embodiments are substantially free, if not totally free, of each of the aforementioned polysorbates, polyethoxylated vegetable oils (whether hydrogenated in whole or in part or not hydrogenated), and substituted cellulosic polymers.

In addition to merely dissolving the docetaxel, the docetaxel (as is or in the presence of TPGS and/or the lipoic acid (or its pharmaceutically acceptable salts) can be lyophilized and presented as a lyophilizate for reconstitution to a concentrate material (of either the initial high concentrate formulation concentrations or directly to the intermediate concentrate formulations or even directly to the administrable concentrations depending on whether the lyophilizate contains the other components required by the diluted concentrate and in the appropriate amounts). However, if these lyophilizates or solid blends of these componentsare used, they are preferably used to prepare an initial concentrate as described herein or used to directly prepare the intermediate concentrateas described herein. The lyophilization procedure can be a routine lyophilization using an appropriate solvent for lyophilization purposes. Insofar as the lyophilization solvent is driven off in the course of the lyophilization procedure, lyophilization may use solvents that are not suitable for parenteral administration, but generally will use suitable materials for parenteral use, so as to avoid potential contamination of the injection product within even minor amounts of injection unsuitable solvents.

Additional components that may be incorporated into the invention formulations include auxiliary aids such protectants against oxidative degradation such as, without limitation, antioxidants and free radical scavengers, such as, without limitation, α-lipoic acid (also known as thioctic acid), its pharmaceutically acceptable salts, dihydrolipoic acid, its pharmaceutically acceptable salts, sulfa amino acids (such as, without limitation, methionine and cysteine), acetone bisulfite and its alkaline salts, ascorbic acid, among others known in the art as suitable for injection purposes. These optional materials are of value as the TPGS component has the potential of being contaminated with a small amount of peroxide molecules formed during its synthesis, which varies from batch to batch. Incorporation of the protectant or free radical/peroxide scavenger protects the docetaxel from oxidative and free radical catalyzed degradative processes that may be caused thereby. When included, the lipoic acid or dihydrolipoic acid (or the salts of either) generally included in the initial concentrate (or even as a blend with the raw docetaxel) but, when desired, may be included in the diluent used to dilute the initial concentrate to make the intermediate concentrate. Applicant has also filed on even date herewith an additional application that is specifically directed to improved lipoic acid and/or dihydrolipoic acid compositions of this nature, which is incorporated herein in its entirety by reference. Exemplary diluent compositions for diluting 1 ml of the initial concentrate (about 80 mg docetaxel/ml and 5 mg/ml lipoic acid) to the diluted concentrate (10 mg docetaxel/ml) are, without limitation,

| | | | | | | |
|---|---|---|---|---|---|---|
| TPGS 1000 | 1.0 | g | 0.75 | g | 0.5 | g |
| Polyethylene glycol | 3.5 | ml | 4.5 | ml | 5 | ml |
| Ethanol | 0.9 | ml | 0.72 | ml | 1 | ml |
| NaCl | 72 | mg | 72 | mg | 72 | mg |
| Water | qs to 7 | ml | qs to 7 | ml | qs to 7 | ml |

A portion of the ethanol may be included in the initial concentrates, but if so, the amount of the ethanol in the diluent formulation is restricted so that upon dilution of the initial concentrate with the diluent formulation, to a concentration of docetaxel of 10 mg/ml, there is preferably not more than a total of 1 ml of ethanol present. When sulfa amino acids are used in place of or in addition to the lipoic acid, they can be used in amounts generally such that the sum of the lipoic acid and the sulfa amino acid amounts (on a molar basis) meet the limitations for the lipoic acid above (based on a molar basis). The remaining alternatives for lipoic acid as set forth above can be used in amounts such that once the formulation is diluted to administration concentrations of docetaxel, the alternative is present in an amount that is suitable for infusions at the resultant concentration AND total infusion dose. These amounts will be known to those of ordinary skill in the intravenous infusion administration art, such as by reference to standard pharmaceutical references as the United States Pharmacopoeia and Remington's Pharmaceutical Sciences.

As the present invention is directed to delivery of docetaxel, once diluted to appropriate injection (especially infusion, most particularly IV infusion) concentrations, it may be administered in appropriate amounts for treating docetaxel responsive conditions known in the art. In addition, since the present invention permits higher doses and concentrations than the currently marketed TAXOTERE, the concentrates and administrable dosage forms thereof made from the present invention are also useful for many of the indications known in the art for docetaxel based on non-clinical data for which the current marketed TAXOTERE formulation is not recommended because of an inability to administer docetaxel at a sufficiently high dose, either acutely or cumulatively. These include, without limitation carcinomas such as colorectal, prostate, pancreatic and liquid tumors like lymphoma and leukemia.

The following examples are presented to exemplify, not limit, the scope of the present invention, which is only limited by the claims appended hereto. In the following examples LA=Lipooic Acid; DCT=docetaxel; TPGS or Vitamin E TPGS=Tocopherol PolyethyleneGlycol Succinate; PEG=polyethyleneglycol.

### EXAMPLE 1

The composition in the table below was diluted with Normal Saline to the indicated docetaxel concentration of either 0.75 mg/ml or 0.32 mg/ml and observed for particulates over the time indicated.

| Composition | Time | 0.75 mg/mL | 0.32 mg/mL |
|---|---|---|---|
| Liquid concentrate: | Observed up | Clear, no | Clear, no |
| DCT - 80 mg; | to 8 hrs | particles | particles |
| LA - 5 mg | | appeared | appeared |
| Glycofurol - qs to | | | |
| 1 mL | | | |
| Diluent: | | | |
| TPGS 1000 - 750 mg | | | |
| PEG 400 - 4.0 mL | | | |
| Ethanol - 0.9 mL | | | |
| Water qs to 7 mL | | | |

### EXAMPLE 2

The composition in the table below was diluted with Normal Saline to the indicated docetaxel concentration of either 0.75 mg/ml or 0.32 mg/ml and observed for particulates over the time indicated.

| Composition | Time | 0.75 mg/mL | 0.32 mg/mL |
|---|---|---|---|
| Liquid concentrate: | Initial | Clear | Clear |
| DCT - 80 mg; LA - | 30 Min | Clear | Clear |
| 5 mg/ | 60 Min | Particles | Clear |
| Glycofurol - qs to | 1 hr 10 min | Particles | Particles |
| 1 mL | | | |
| Diluent: | | | |
| TPGS 1000 - 750 mg | | | |
| PEG 400 - 2.5 mL | | | |
| Ethanol - 0.9 mL | | | |
| Water qs to 7 mL | | | |

### EXAMPLES 3-4

The composition in the table below was diluted with Normal Saline to the indicated docetaxel concentration of either 0.75 mg/ml or 0.32 mg/ml and observed for particulates over the time indicated.

| | | Label | | Content | |
|---|---|---|---|---|---|
| | | Claim | Time | (mg/mL) | |
| Example | Composition | (mg/mL) | period | at ART | Physical Observation |
| Example 3 | DCT - 80 mg | 0.32 | Initial | 0.34 | Particles appearance |
| | Glycofurol - 1 mL | | 8 hr | 0.31 | started after 5.30 hr |
| | Diluent | 0.74 | Initial | 0.74 | Particles appearance |
| | Vit. E TPGS - 750 mg | | 8 hr | 0.73 | started after 5.00 hr |
| | PEG-400 - 6 mL | | | | |
| | (No Ethanol &) | | | | |
| | Water qs to 7 mL | | | | |
| Example 4 | DCT - 80 mg | 0.32 | Initial | 0.33 | Particles appearance |
| | Glycofurol - 1 mL | | 8 hr | 0.31 | started after 7.00 hr |
| | Diluent | 0.74 | Initial | 0.76 | Particles appearance |
| | Vit. E TPGS - 500 mg | | 8 hr | 0.58 | started after 7.00 hr |
| | Ethanol - 1 mL | | | | |
| | PEG-400 - 5 mL | | | | |
| | Water - qs to 7 mL | | | | |

### EXAMPLE 5

Compositions of docetaxel liquid concentrate containing a total amount of 80 mg of docetaxel are prepared having the compositions shown in the following table. In each case, the antioxidant is chosen from (a) lipoic acid, (b) dihyrolipoic acid, (c) lipoic acid sodium salt, (d) lipoic acid potassium salt, (e) dihydrolipoic acid sodium salt, and (f) dihydrolipoic acid potassium salt, and each composition no. indicates a series of these six variants

| Composition # | Glycofurol | Ethanol | | Antioxidant |
|---|---|---|---|---|
| 1 | 1 ml | 0 | | 2.5 mg |
| 2 | 1 ml | 0 | | 5.0 mg |
| 3 | 1 ml | 0 | | 7.5 mg |
| 4 | 1 ml | 0 | | 10.0 mg |
| 5 | 1 ml | 0 | | 12.5 mg |
| 6 | 1 ml | 0 | | 15.0 mg |
| 7 | 1 ml | 0 | | 17.5 mg |
| 8 | 1 ml | 0 | | 20.0 mg |
| 9 | 0.5 ml | 0.5 | ml | 2.5 mg |
| 10 | 0.5 ml | 0.5 | ml | 5.0 mg |
| 11 | 0.5 ml | 0.5 | ml | 7.5 mg |
| 12 | 0.5 ml | 0.5 | ml | 10.0 mg |
| 13 | 0.5 ml | 0.5 | ml | 12.5 mg |
| 14 | 0.5 ml | 0.5 | ml | 15.0 mg |
| 15 | 0.5 ml | 0.5 | ml | 17.5 mg |
| 16 | 0.5 ml | 0.5 | ml | 20.0 mg |
| 17 | 0.75 ml | 0.75 | ml | 2.5 mg |
| 18 | 0.75 ml | 0.75 | ml | 5.0 mg |
| 19 | 0.75 ml | 0.75 | ml | 7.5 mg |
| 20 | 0.75 ml | 0.75 | ml | 10.0 mg |
| 21 | 0.75 ml | 0.75 | ml | 12.5 mg |
| 22 | 0.75 ml | 0.75 | ml | 15.0 mg |
| 23 | 0.75 ml | 0.75 | ml | 17.5 mg |
| 24 | 0.75 ml | 0.75 | ml | 20.0 mg |
| 25 | 1 ml | 1 | ml | 2.5 mg |
| 26 | 1 ml | 1 | ml | 5.0 mg |
| 27 | 1 ml | 1 | ml | 7.5 mg |
| 28 | 1 ml | 1 | ml | 10.0 mg |
| 29 | 1 ml | 1 | ml | 12.5 mg |
| 30 | 1 ml | 1 | ml | 15.0 mg |
| 31 | 1 ml | 1 | ml | 17.5 mg |
| 32 | 1 ml | 1 | ml | 20.0 mg |

### EXAMPLE 6

Compositions of the diluent for compositions 1-8 of Example 5

| PEG 400 (ml) | Ethanol (ml) | TPGS-1000 (mg) | NaCl (mg) | Water QS |
|---|---|---|---|---|
| 4 | 0.75 | 500 | 50 | 7 |
| 4 | 0.75 | 750 | 50 | 7 |
| 4 | 0.75 | 1000 | 50 | 7 |
| 5 | 0.75 | 500 | 50 | 7 |
| 5 | 0.75 | 750 | 50 | 7 |
| 5 | 0.75 | 1000 | 50 | 7 |
| 6 | 0.75 | 500 | 50 | 7 |
| 6 | 0.75 | 750 | 50 | 7 |
| 6 | 0.75 | 1000 | 50 | 7 |
| 4 | 1.00 | 500 | 50 | 7 |
| 4 | 1.00 | 750 | 50 | 7 |
| 4 | 1.00 | 1000 | 50 | 7 |
| 5 | 1.00 | 500 | 50 | 7 |
| 5 | 1.00 | 750 | 50 | 7 |
| 5 | 1.00 | 1000 | 50 | 7 |
| 6 | 1.00 | 500 | 50 | 7 |
| 6 | 1.00 | 750 | 50 | 7 |
| 6 | 1.00 | 1000 | 50 | 7 |
| 4 | 0.75 | 500 | 65 | 7 |
| 4 | 0.75 | 750 | 65 | 7 |
| 4 | 0.75 | 1000 | 65 | 7 |
| 5 | 0.75 | 500 | 65 | 7 |
| 5 | 0.75 | 750 | 65 | 7 |
| 5 | 0.75 | 1000 | 65 | 7 |
| 6 | 0.75 | 500 | 65 | 7 |
| 6 | 0.75 | 750 | 65 | 7 |
| 6 | 0.75 | 1000 | 65 | 7 |
| 4 | 1.00 | 500 | 65 | 7 |
| 4 | 1.00 | 750 | 65 | 7 |
| 4 | 1.00 | 1000 | 65 | 7 |
| 5 | 1.00 | 500 | 65 | 7 |
| 5 | 1.00 | 750 | 65 | 7 |
| 5 | 1.00 | 1000 | 65 | 7 |
| 6 | 1.00 | 500 | 65 | 7 |
| 6 | 1.00 | 750 | 65 | 7 |
| 6 | 1.00 | 1000 | 65 | 7 |
| 4 | 0.75 | 500 | 72 | 7 |
| 4 | 0.75 | 750 | 72 | 7 |
| 4 | 0.75 | 1000 | 72 | 7 |
| 5 | 0.75 | 500 | 72 | 7 |
| 5 | 0.75 | 750 | 72 | 7 |
| 5 | 0.75 | 1000 | 72 | 7 |
| 6 | 0.75 | 500 | 72 | 7 |
| 6 | 0.75 | 750 | 72 | 7 |
| 6 | 0.75 | 1000 | 72 | 7 |
| 4 | 1.00 | 500 | 72 | 7 |
| 4 | 1.00 | 750 | 72 | 7 |
| 4 | 1.00 | 1000 | 72 | 7 |
| 5 | 1.00 | 500 | 72 | 7 |
| 5 | 1.00 | 750 | 72 | 7 |
| 5 | 1.00 | 1000 | 72 | 7 |
| 6 | 1.00 | 500 | 72 | 7 |
| 6 | 1.00 | 750 | 72 | 7 |
| 6 | 1.00 | 1000 | 72 | 7 |
| 4 | 0.75 | 500 | 75 | 7 |
| 4 | 0.75 | 750 | 75 | 7 |
| 4 | 0.75 | 1000 | 75 | 7 |
| 5 | 0.75 | 500 | 75 | 7 |
| 5 | 0.75 | 750 | 75 | 7 |
| 5 | 0.75 | 1000 | 75 | 7 |
| 6 | 0.75 | 500 | 75 | 7 |
| 6 | 0.75 | 750 | 75 | 7 |
| 6 | 0.75 | 1000 | 75 | 7 |
| 4 | 1.00 | 500 | 75 | 7 |
| 4 | 1.00 | 750 | 75 | 7 |
| 4 | 1.00 | 1000 | 75 | 7 |
| 5 | 1.00 | 500 | 75 | 7 |
| 5 | 1.00 | 750 | 75 | 7 |
| 5 | 1.00 | 1000 | 75 | 7 |
| 6 | 1.00 | 500 | 75 | 7 |
| 6 | 1.00 | 750 | 75 | 7 |
| 6 | 1.00 | 1000 | 75 | 7 |

### EXAMPLE 7

Compositions of the diluent for compositions 9-16 of Example 5

| PEG 400 (ml) | Ethanol (ml) | TPGS-1000 (mg) | NaCl (mg) | Water QS |
|---|---|---|---|---|
| 4 | 0.25 | 500 | 50 | 7 |
| 4 | 0.25 | 750 | 50 | 7 |
| 4 | 0.25 | 1000 | 50 | 7 |
| 5 | 0.25 | 500 | 50 | 7 |
| 5 | 0.25 | 750 | 50 | 7 |
| 5 | 0.25 | 1000 | 50 | 7 |
| 6 | 0.25 | 500 | 50 | 7 |
| 6 | 0.25 | 750 | 50 | 7 |
| 6 | 0.25 | 1000 | 50 | 7 |
| 4 | 0.25 | 500 | 65 | 7 |
| 4 | 0.25 | 750 | 65 | 7 |
| 4 | 0.25 | 1000 | 65 | 7 |
| 5 | 0.25 | 500 | 65 | 7 |
| 5 | 0.25 | 750 | 65 | 7 |
| 5 | 0.25 | 1000 | 65 | 7 |
| 6 | 0.25 | 500 | 65 | 7 |
| 6 | 0.25 | 750 | 65 | 7 |
| 6 | 0.25 | 1000 | 65 | 7 |
| 4 | 0.25 | 500 | 72 | 7 |
| 4 | 0.25 | 750 | 72 | 7 |
| 4 | 0.25 | 1000 | 72 | 7 |
| 5 | 0.25 | 500 | 72 | 7 |
| 5 | 0.25 | 750 | 72 | 7 |
| 5 | 0.25 | 1000 | 72 | 7 |
| 6 | 0.25 | 500 | 72 | 7 |
| 6 | 0.25 | 750 | 72 | 7 |
| 6 | 0.25 | 1000 | 72 | 7 |
| 4 | 0.25 | 500 | 75 | 7 |
| 4 | 0.25 | 750 | 75 | 7 |
| 4 | 0.25 | 1000 | 75 | 7 |
| 5 | 0.25 | 500 | 75 | 7 |
| 5 | 0.25 | 750 | 75 | 7 |
| 5 | 0.25 | 1000 | 75 | 7 |
| 6 | 0.25 | 500 | 75 | 7 |
| 6 | 0.25 | 750 | 75 | 7 |
| 6 | 0.25 | 1000 | 75 | 7 |
| 4 | 0.50 | 500 | 50 | 7 |
| 4 | 0.50 | 750 | 50 | 7 |
| 4 | 0.50 | 1000 | 50 | 7 |
| 5 | 0.50 | 500 | 50 | 7 |
| 5 | 0.50 | 750 | 50 | 7 |
| 5 | 0.50 | 1000 | 50 | 7 |
| 6 | 0.50 | 500 | 50 | 7 |
| 6 | 0.50 | 750 | 50 | 7 |
| 6 | 0.50 | 1000 | 50 | 7 |
| 4 | 0.50 | 500 | 65 | 7 |
| 4 | 0.50 | 750 | 65 | 7 |
| 4 | 0.50 | 1000 | 65 | 7 |
| 5 | 0.50 | 500 | 65 | 7 |
| 5 | 0.50 | 750 | 65 | 7 |
| 5 | 0.50 | 1000 | 65 | 7 |
| 6 | 0.50 | 500 | 65 | 7 |
| 6 | 0.50 | 750 | 65 | 7 |
| 6 | 0.50 | 1000 | 65 | 7 |
| 4 | 0.50 | 500 | 72 | 7 |
| 4 | 0.50 | 750 | 72 | 7 |
| 4 | 0.50 | 1000 | 72 | 7 |
| 5 | 0.50 | 500 | 72 | 7 |
| 5 | 0.50 | 750 | 72 | 7 |
| 5 | 0.50 | 1000 | 72 | 7 |
| 6 | 0.50 | 500 | 72 | 7 |
| 6 | 0.50 | 750 | 72 | 7 |
| 6 | 0.50 | 1000 | 72 | 7 |
| 4 | 0.50 | 500 | 75 | 7 |
| 4 | 0.50 | 750 | 75 | 7 |
| 4 | 0.50 | 1000 | 75 | 7 |
| 5 | 0.50 | 500 | 75 | 7 |
| 5 | 0.50 | 750 | 75 | 7 |
| 5 | 0.50 | 1000 | 75 | 7 |
| 6 | 0.50 | 500 | 75 | 7 |
| 6 | 0.50 | 750 | 75 | 7 |
| 6 | 0.50 | 1000 | 75 | 7 |

### EXAMPLE 8

Compositions of the diluent for compositions 17-24 of Example 5

| PEG 400 (ml) | Ethanol (ml) | TPGS-1000 (mg) | NaCl (mg) | Water QS |
|---|---|---|---|---|
| 4 | 0.1 | 500 | 50 | 6.5 |
| 4 | 0.1 | 750 | 50 | 6.5 |
| 4 | 0.1 | 1000 | 50 | 6.5 |
| 5 | 0.1 | 500 | 50 | 6.5 |
| 5 | 0.1 | 750 | 50 | 6.5 |
| 5 | 0.1 | 1000 | 50 | 6.5 |
| 6 | 0.1 | 500 | 50 | 6.5 |
| 6 | 0.1 | 750 | 50 | 6.5 |
| 6 | 0.1 | 1000 | 50 | 6.5 |
| 4 | 0.1 | 500 | 65 | 6.5 |
| 4 | 0.1 | 750 | 65 | 6.5 |
| 4 | 0.1 | 1000 | 65 | 6.5 |
| 5 | 0.1 | 500 | 65 | 6.5 |
| 5 | 0.1 | 750 | 65 | 6.5 |
| 5 | 0.1 | 1000 | 65 | 6.5 |
| 6 | 0.1 | 500 | 65 | 6.5 |
| 6 | 0.1 | 750 | 65 | 6.5 |
| 6 | 0.1 | 1000 | 65 | 6.5 |
| 4 | 0.1 | 500 | 72 | 6.5 |
| 4 | 0.1 | 750 | 72 | 6.5 |
| 4 | 0.1 | 1000 | 72 | 6.5 |
| 5 | 0.1 | 500 | 72 | 6.5 |
| 5 | 0.1 | 750 | 72 | 6.5 |
| 5 | 0.1 | 1000 | 72 | 6.5 |
| 6 | 0.1 | 500 | 72 | 6.5 |
| 6 | 0.1 | 750 | 72 | 6.5 |
| 6 | 0.1 | 1000 | 72 | 6.5 |
| 4 | 0.1 | 500 | 75 | 6.5 |
| 4 | 0.1 | 750 | 75 | 6.5 |
| 4 | 0.1 | 1000 | 75 | 6.5 |
| 5 | 0.1 | 500 | 75 | 6.5 |
| 5 | 0.1 | 750 | 75 | 6.5 |
| 5 | 0.1 | 1000 | 75 | 6.5 |
| 6 | 0.1 | 500 | 75 | 6.5 |
| 6 | 0.1 | 750 | 75 | 6.5 |
| 6 | 0.1 | 1000 | 75 | 6.5 |
| 4 | 0.25 | 500 | 50 | 6.5 |
| 4 | 0.25 | 750 | 50 | 6.5 |
| 4 | 0.25 | 1000 | 50 | 6.5 |
| 5 | 0.25 | 500 | 50 | 6.5 |
| 5 | 0.25 | 750 | 50 | 6.5 |
| 5 | 0.25 | 1000 | 50 | 6.5 |
| 6 | 0.25 | 500 | 50 | 6.5 |
| 6 | 0.25 | 750 | 50 | 6.5 |
| 6 | 0.25 | 1000 | 50 | 6.5 |
| 4 | 0.25 | 500 | 65 | 6.5 |
| 4 | 0.25 | 750 | 65 | 6.5 |
| 4 | 0.25 | 1000 | 65 | 6.5 |
| 5 | 0.25 | 500 | 65 | 6.5 |
| 5 | 0.25 | 750 | 65 | 6.5 |
| 5 | 0.25 | 1000 | 65 | 6.5 |
| 6 | 0.25 | 500 | 65 | 6.5 |
| 6 | 0.25 | 750 | 65 | 6.5 |
| 6 | 0.25 | 1000 | 65 | 6.5 |
| 4 | 0.25 | 500 | 72 | 6.5 |
| 4 | 0.25 | 750 | 72 | 6.5 |
| 4 | 0.25 | 1000 | 72 | 6.5 |
| 5 | 0.25 | 500 | 72 | 6.5 |
| 5 | 0.25 | 750 | 72 | 6.5 |
| 5 | 0.25 | 1000 | 72 | 6.5 |
| 6 | 0.25 | 500 | 72 | 6.5 |
| 6 | 0.25 | 750 | 72 | 6.5 |
| 6 | 0.25 | 1000 | 72 | 6.5 |
| 4 | 0.1-0.5 | 500 | 72 | 6.5 |
| 4 | 0.25 | 750 | 72 | 6.5 |
| 4 | 0.25 | 1000 | 72 | 6.5 |
| 5 | 0.25 | 500 | 72 | 6.5 |
| 5 | 0.25 | 750 | 72 | 6.5 |
| 5 | 0.25 | 1000 | 72 | 6.5 |
| 6 | 0.25 | 500 | 72 | 6.5 |
| 6 | 0.25 | 750 | 72 | 6.5 |
| 6 | 0.25 | 1000 | 72 | 6.5 |
| 4 | 0.25 | 500 | 75 | 6.5 |
| 4 | 0.25 | 750 | 75 | 6.5 |
| 4 | 0.25 | 1000 | 75 | 6.5 |
| 5 | 0.25 | 500 | 75 | 6.5 |
| 5 | 0.25 | 750 | 75 | 6.5 |
| 5 | 0.25 | 1000 | 75 | 6.5 |
| 6 | 0.25 | 500 | 75 | 6.5 |
| 6 | 0.25 | 750 | 75 | 6.5 |
| 6 | 0.25 | 1000 | 75 | 6.5 |

### EXAMPLE 9

Compositions of the diluent for compositions 25-32 of Example 5

| PEG 400 (ml) | Ethanol (ml) | TPGS-1000 (mg) | NaCl (mg) | Water QS |
|---|---|---|---|---|
| 4 | 0.0 | 500 | 50 | 6.0 |
| 4 | 0.0 | 750 | 50 | 6.0 |
| 4 | 0.0 | 1000 | 50 | 6.0 |
| 5 | 0.0 | 500 | 50 | 6.0 |
| 5 | 0.0 | 750 | 50 | 6.0 |
| 5 | 0.0 | 1000 | 50 | 6.0 |
| 6 | 0.0 | 500 | 50 | 6.0 |
| 6 | 0.0 | 750 | 50 | 6.0 |
| 6 | 0.0 | 1000 | 50 | 6.0 |
| 4 | 0.0 | 500 | 65 | 6.0 |
| 4 | 0.0 | 750 | 65 | 6.0 |
| 4 | 0.0 | 1000 | 65 | 6.0 |
| 5 | 0.0 | 500 | 65 | 6.0 |
| 5 | 0.0 | 750 | 65 | 6.0 |
| 5 | 0.0 | 1000 | 65 | 6.0 |
| 6 | 0.0 | 500 | 65 | 6.0 |
| 6 | 0.0 | 750 | 65 | 6.0 |
| 6 | 0.0 | 1000 | 65 | 6.0 |
| 4 | 0.0 | 500 | 72 | 6.0 |
| 4 | 0.0 | 750 | 72 | 6.0 |
| 4 | 0.0 | 1000 | 72 | 6.0 |
| 5 | 0.0 | 500 | 72 | 6.0 |
| 5 | 0.0 | 750 | 72 | 6.0 |
| 5 | 0.0 | 1000 | 72 | 6.0 |
| 6 | 0.0 | 500 | 72 | 6.0 |
| 6 | 0.0 | 750 | 72 | 6.0 |
| 6 | 0.0 | 1000 | 72 | 6.0 |
| 4 | 0.0 | 500 | 75 | 6.0 |
| 4 | 0.0 | 750 | 75 | 6.0 |
| 4 | 0.0 | 1000 | 75 | 6.0 |
| 5 | 0.0 | 500 | 75 | 6.0 |
| 5 | 0.0 | 750 | 75 | 6.0 |
| 5 | 0.0 | 1000 | 75 | 6.0 |
| 6 | 0.0 | 500 | 75 | 6.0 |
| 6 | 0.0 | 750 | 75 | 6.0 |
| 6 | 0.0 | 1000 | 75 | 6.0 |

We claim:

1. An initial concentrated docetaxel solution comprising
(a) a first component selected from the group consisting of docetaxel a pharmaceutically acceptable salt thereof, and mixtures thereof;
(b) a second component being a solvent for said first component selected from the group consisting of glycofurol and ethanol, and mixtures thereof;
(c) optionally a third component which is selected from the group consisting of α-lipoic acid, dihydrolipoic acid, pharmaceutically acceptable salts of either, and mixtures thereof;
(d) optionally a fourth component which is polyethylene glycol 400;
(e) optionally a fifth component selected from the group consisting of tonicity adjusting agents;
(f) optionally a sixth component selected from additional antioxidant which additional antioxidant is other than that of said third component;
(g) and optionally water;
said concentrated docetaxel formulation being substantially free of polysorbate surfactants, and polyethoxylated forms of a member selected from castor oil, partially hydrogenated castor oil, fully hydrogenated castor oil, vegetable oil, partially hydrogenated vegetable oil, and fully hydrogenated vegetable oil.

2. The initial concentrate of claim 1 wherein said docetaxel concentration is at least in excess of 10 mg up to 160 mg docetaxel or pharmaceutically acceptable salt thereof (based on free docetaxel) per ml of said initial concentrate.

3. The initial concentrate of claim 1 wherein said solvent is at least a glycofurol alone or in admixture with ethanol.

4. The initial concentrate of claim 1 wherein said third component is present and in an amount of at least more than 2 mg up to not more than 20 mg (based on the non-salt forms respectively)/ml of said initial concentrate.

5. The initial concentrate of claim 1 wherein said third component is present and in an amount of at about 5 mg (based on a non-salt form thereof respectively)/ml of said initial concentrate.

6. A diluent formulation for use in the dilution of a docetaxel containing initial concentrate solution of claim 1 to an intermediate concentration solution, said intermediate concentration having docetaxel or a pharmaceutically acceptable salt thereof in an amount of not greater than 15 mg docetaxel/ml of said intermediate concentration solution; said diluent formulation comprising:
(a) a tocopherol polyethylene glycol succinate (TPGS);
(b) optionally an initial antioxidant member selected from the group consisting of α-lipoic acid, dihydrolipoic acid, pharmaceutically acceptable salts of either, and mixtures thereof;
(c) optionally a polyethylene glycol;
(d) optionally ethanol;
(e) optionally a tonicity adjusting agent;
(f) optionally additional antioxidant; and
(g) water;
such that upon dilution of said initial concentrate solution with said diluentformulation,
said TPGS is present in an amount of from >0 up to 18 parts by weight per part by weight of docetaxel or pharmaceutically salt thereof based on free docetaxel;
and said ethanol is present and in a total amount of up to 0.016875 ml per mg docetaxel or pharmaceutically acceptable salt thereof based on free docetaxel;
said initial antioxidant being present in an amount in said diluent formulation up to an amount such that on combination of said diluent formulation with said initial concentrate, said initial antioxidant is present in an amount of from greater than 2 mg to less than 200 mg;
said polyethylene glycol is present in an amount of from greater than 0.03125 ml to not more than 0.75 ml/mg of docetaxel or pharmaceutically acceptable salt thereof based on free docetaxel;
said tonicity agent is optionally present up to an amount of up to an ionic strength equal to that of 100 mg NaCl/ml of said intermediate concentration.

7. A docetaxel containing high concentration liquid solution comprising
(a) docetaxel or a pharmaceutically acceptable salt thereof in a concentration in excess of 10 mg (based on docetaxel)/ml of said high concentration liquid solution;
(b) glycofurol;
(c) ethanol in an amount of up to 0.016875 ml per mg docetaxel or pharmaceutically acceptable salt thereof based on docetaxel; and
(d) a component which is selected from the group consisting of α-lipoic acid, dihydrolipoic acid, pharmaceutically acceptable salts of either, and mixtures thereof in an amount of more than 2 mg to less 200 mg.

8. The docetaxel containing high concentration liquid solution of claim 7 wherein said ethanol is present in an amount of from 0.005625 ml to 0.016875 ml per mg docetaxel or pharmaceutically acceptable salt thereof based on docetaxel.

9. The docetaxel containing high concentration liquid solution of claim 7 wherein said ethanol is present in an amount of from 0.00875 ml to 0.01375 ml per mg docetaxel or pharmaceutically acceptable salt thereof based on docetaxel.

10. The docetaxel containing high concentration liquid solution of claim 7 wherein said ethanol is present in an amount of from 0.01 ml to 0.0125 ml per mg docetaxel or pharmaceutically acceptable salt thereof based on docetaxel.

11. The docetaxel containing high concentration liquid solution of claim 7 wherein said ethanol is present in an amount of about 0.01125 ml per mg docetaxel or pharmaceutically acceptable salt thereof based on docetaxel.

12. A docetaxel infusion comprising
(a) docetaxel or a pharmaceutically acceptable salt thereof in an infusion suitable amount
(b) glycofurol;
(c) tocopherol polyethylene glycol succinate (TPGS) in an amount of from >0 up to 18 parts by weight per part by weight of said docetaxel or pharmaceutically acceptable salt thereof based on docetaxel;
(d) polyethylene glycol;
(e) ethanol in an amount greater than 0 and not more than 0.016875 ml per mg docetaxel or pharmaceutically acceptable salt thereof based on docetaxel;
(f) optionally a tonicity adjusting agent;
(g) optionally a component which is selected from the group consisting of α-lipoic acid, dihydrolipoic acid, pharmaceutically acceptable salts of either, and mixtures thereof;
(h) optionally antioxidizing agent other than component (g) above;
(i) water; and
further infusion fluids or infusion liquid formulations having components compatible with the foregoing components (a)-(i).

13. A method of enhancing the stability of a docetaxel liquid formulation, said formulation comprising at least docetaxel and tocopherol polyethylene glycol succinate (TPGS), said method comprising limiting the ratio of said TPGS to not more than 18 parts by weight tocopherol polyethylene glycol succinate (TPGS) per part by weight of said docetaxel or docetaxel portion of said pharmaceutically acceptable salt of said docetaxel.

14. A method of extending the time period between (a) dilution of a docetaxel containing high concentrate solution to a less concentrated docetaxol containing concentrate for further dilution to an infusion and (b) when the infusion administration must be completed from not more than 4 hours to a period in substantial excess of 4 hours, said method comprising preparing said less concentrated docetaxel containing concentrate wherein said less concentrated concentrate comprises
(a) docetaxel or a pharmaceutically acceptable slat thereof in an amount of less than 15 mg (based on docetaxel) per ml of said less concentrated concentrate;
(b) glycofurol;
(c) tocopherol polyethylene glycol succinate in an amount of >0 up to 18 parts by weight TPGS per part by weight of said docetaxel or pharmaceutically acceptable salt thereof based on docetaxel;
(d) polyethylene glycol
(e) ethanol in an amount greater than 0 and not more than 0.016875 ml per mg docetaxel or pharmaceutically acceptable salt thereof based on docetaxel;
(f) optionally a tonicity adjuster;
(g) a component which is selected from the group consisting of α-lipoic acid, dihydrolipoic acid, pharmaceutically acceptable salts of either, and mixtures thereof;
(h) optionally antioxidizing agent other than component (g) above; and
(i) water;
where said so prepared less concentrated docetaxel concentrate is used in an infusion administration procedure.

15. A method of treating a docetaxel responsive condition in an animal in need of the same comprising administering a docetaxel containing infusion comprising
(a) docetaxel in an infusion suitable concentration;
(b) glycofurol;
(c) a member selected from the group consisting of α-lipoic acid, dihydrolipoic acid, pharmaceutically acceptable salts of either, and mixtures thereof;
(d) tocopherol polyethyleneglycol succinate;
(e) polyethylene glycol;
(f) optionally ethanol;
(g) optionally a tonicity adjuster;and
(h) an infusion fluid suitable for administration to said animal.

16. The method of claim 15 wherein hypersensitivity reactions seen with Taxotere that require pre-medication with a member selected from antihistamines, steroids, or both, are reduced relative to Taxotere to an extent that either of both of said antihistamine and/or steroid pre-treatment can be omitted or substantially reduced.

17. A method of avoiding pre-treatment with (a) an antihistamine or (b) a steroid, or (c) both in an animal in need of a docetaxel (or pharmaceutically acceptable salt thereof) infusion administration comprising administering to said animal a docetaxel (or pharmaceutically acceptable salt thereof) infusion according to claim 12.

18. A method of avoiding gastrointestinal side effect seen with Taxotere in an animal in need of a docetaxel or pharmaceutically acceptable salt thereof) infusion administration comprising administering to said animal a docetaxel (or pharmaceutically acceptable salt thereof) infusion according to claim 12.
